(19) United States
(12) Patent Application Publication
(10) Pub. No.: US 2017/0029781 A1
(43) Pub. Date: Feb. 2, 2017

(54) METHOD FOR PREPARING ARTIFICIAL BLOOD VESSELS

(71) Applicant: 3D GLOBAL BIOTECH INC., Taipei City (TW)

(72) Inventor: Keng-Liang OU, Kaohsiung City (TW)

(21) Appl. No.: 15/175,198

(22) Filed: Jun. 7, 2016

(30) Foreign Application Priority Data
Jul. 31, 2015 (TW) 104124823

(51) Int. Cl. C12N 5/071; A61F 2/06; C12N 13/00

(52) U.S. Cl. CPC C12N 5/0691; C12N 13/00; A61F 2/06

(57) ABSTRACT

A method for preparing artificial vessels comprises preparing a template by 3D printing; preparing an active polyhedral oligomeric silsesquioxane poly(carbonate-urea) urethane (active POSS-PCU); mixing the active POSS-PCU with stem cells to form the artificial vessels by 3D printing followed by plasma processing; removing the template to form the artificial blood vessels with an access. The method provides to prepare the artificial vessels with three-layer structures, which are capable of transporting nutrients and oxygen, removing metabolic wastes and enhancing haemocompatibility and biological stability. Therefore, the method for preparing the artificial vessels solves the problem of angiemphraxis caused by the artificial vessels with single-layer structure.

### BACKGROUND OF THE INVENTION

1. Field of the Invention

The present invention relates to a method for preparing artificial blood vessels, especially to a method for preparing artificial blood vessels with an active POSS-PCU and stem cells.

2. Description of the Prior Art

The conventional method for preparing artificial blood vessels comprises using biomaterials, such as silica gel or polytetrafluoroethylene (PTFE). The artificial blood vessels prepared by PTFE have been applied to brain surgery and heart bypass operation. Another conventional method for preparing the artificial blood vessels comprises coating a hollow column comprising GoreTex® biomaterials with a blood capsule containing cells. The cells would grow on the hollow column comprising GoreTex® biomaterials, allowing the GoreTex® biomaterials to be replaced with the cells. Another conventional method for preparing artificial blood vessels comprises: preparing a template with agarose fiber by 3D printing, and enveloping the template with biomaterials or with biomaterials mixed with cells to form artificial blood vessels; enhancing the strength of the artificial blood vessels by photocrosslink technology; finally removing the template to form the artificial blood vessels with an access.

However, the artificial blood vessels prepared by the preceding conventional methods merely comprise one single layer. In other words, artificial blood vessels that remodel three layers of the human blood vessels cannot be prepared by the conventional methods. Therefore, the biological stability and functions of the artificial blood vessels with one single layer would be affected in application. Besides, angiemphraxis phenomenon would occur once tissues or thrombi are formed on the inner walls of the artificial blood vessels less than 3 millimeter (mm) in diameter, making the artificial blood vessels unable to transport nutrients and oxygen and remove metabolic wastes. Therefore, the artificial blood vessels less than 3 mm in diameter have not been applied to clinical treatment. Consequently, preparing artificial blood vessels that are less than 3 mm in diameter and with three layers for transporting nutrients and oxygen, removing metabolic wastes and enhancing haemocompatibility and biological stability is important in the present field.

### SUMMARY OF THE INVENTION

To overcome the shortcomings, the present invention provides a method for preparing artificial blood vessels to mitigate or obviate the aforementioned problems.

The method for preparing artificial blood vessels in accordance with the present invention comprises steps of:
preparing a template by 3D printing;
preparing an active polyhedral oligomeric silsesquioxane poly(carbonate-urea) urethane (active POSS-PCU);
preparing stem cells;
mixing the active POSS-PCU with the 1×10⁶ to 2×10⁶ stem cells to form a mixed material containing the stem cells at 4° C.; enveloping the template with the mixed material, and sequentially forming the artificial blood vessels with three layers in a sequentially order away from the template; and
processing the artificial blood vessels with three layers by plasma processing to enhance the structure of the artificial blood vessels; removing the template to form the artificial blood vessels with an access.

Preferably, the source of the stem cells comprises bone marrow or peripheral blood.

Preferably, in the step of preparing stem cells, the stem cells express cell surface antigen CD133 or CD34.

Preferably, in the step of mixing the active POSS-PCU with the stem cells, the active POSS-PCU is mixed with 1×10⁶ stem cells.

Preferably, the conditions of the plasma processing comprise: frequency of DC-discharge plasma 0 Hz, frequency of low/medium-discharge plasma between 10 kilohertz (kHz) and 100 kHz; frequency of radio frequency-discharge plasma 13.56 megahertz (MHz); frequency of microwave-discharge plasma 2.45 gigahertz (GHz); gas flow rate between 0.1 standard liter per minute and 10 standard liters per minute; working power between 1 kilovolt and 40 kilovolts; working power between 1 watt and 180 watts, and reaction time between 5 seconds and 420 seconds.

Compared to the conventional method merely for preparing the artificial vessels with one single layer, the method for preparing the artificial vessels in accordance with the present invention is processed by mixing the active POSS-PCU with the stem cells, and forming the artificial vessels with three layers by 3D printing and differentiation of the stem cells. The artificial vessels are able to transport nutrients and oxygen, remove metabolic wastes and enhance haemocompatibility and biological stability of the artificial vessels. The method for preparing the artificial vessels in accordance with the present invention solves the problem of angiemphraxis caused by the artificial vessels with one single layer prepared by the conventional method, and the artificial vessels with three layers prepared in accordance with the present invention can be applied to brain surgeries, heart bypass operations or treatment of coronary artery tumor diseases, and organ reconstruction of skin, heart, liver and breast.

### BRIEF DESCRIPTION OF THE DRAWINGS

FIG. 1 is a flow chart of a method for preparing artificial blood vessels in accordance with the present invention.

### DETAILED DESCRIPTION OF THE PREFERRED EMBODIMENTS

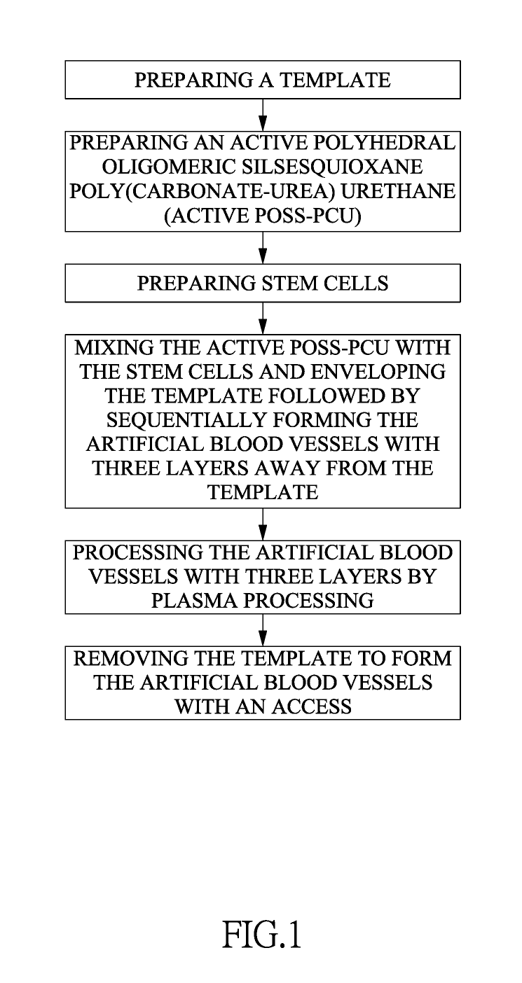

With reference to FIG. 1, a method for preparing artificial blood vessels in accordance with the present invention is shown as follows.

An agarose fiber was formed with liquid agarose by 3D printing at 80° C., and the agarose fiber was then placed at 4° C. to be solidified and formed as a template. Polycarbonate polyol was mixed with trans-cyclohexanediolisobutyl-silsesquioxane at a specific volume ratio under helium gas (He₂) condition. The volume ratio of polycarbonate polyol to trans-cyclohexanediolisobutyl-silsesquioxane was 1 vol %:30 vol % in a volume percentage basis of polycarbonate polyol followed by heating for 5 minutes to 10 minutes at 125° C., allowing the polycarbonate polyol and trans-cyclohexanediolisobutyl-silsesquioxane to be dissolved and mixed as a mixture. The temperature of the mixture was decreased to 60° C. and mixed with diphenyl-methane-diisocyanate (MDI) at a volume ratio of 1 vol %:4 vol % to form a composition. The composition was heated to 80° C. and allowed to stand for 90 minutes followed by mixing the composition with dimethylacetamide. The temperature of the composition mixed with the dimethylacetamide was decreased to 40° C. to form a polyhedral oligomeric silsesquioxane poly(carbonate-urea) urethane (POSS-PCU). The POSS-PCU was kept at 40° C. for remaining at the liquid state. The POSS-PCU was heated to 65° C. to activate peptides to form an active POSS-PCU. Stem cells were separated from bone marrow or peripheral blood with CliniMACS®. In a preferred experiment, the stem cells express surface antigen CD133 or CD34. The active POSS-PCU was mixed with the 1×10⁶ stem cells expressing surface antigen CD133 or CD34 to form a mixed material containing the stem cells at 4° C. The template with the mixed material was enveloped for 3 times by 3D printing. Sequentially, the artificial blood vessels with three layers were formed with the three layers in a sequential order away from the template. The three layers of the artificial blood vessels were confirmed with an image photographed by a computerized tomography. The artificial blood vessels with three layers were processed by plasma processing to enhance the structure of the artificial blood vessels at 15° C. to 30° C. under 760 torr, oxygen (O₂) and He₂ conditions. The conditions of the plasma processing comprise: frequency of DC-discharge plasma 0 Hz, frequency of low/medium-discharge plasma between 10 kHz and 100 kHz; frequency of radio frequency-discharge plasma 13.56 MHz; frequency of microwave-discharge plasma 2.45 GHz; gas flow rate between 0.1 standard liter per minute and 10 standard liters per minute; working power between 1 kilovolt and 40 kilovolts; working power between 1 watt and 180 watts, and reaction time between 5 seconds and 420 seconds. The template was removed to form the artificial blood vessels with an access.

The artificial blood vessels made by the method in accordance with the present invention comprise a first layer, a second layer and a third layer formed in a sequential order from inner to outer of the artificial blood vessels. In a preferred embodiment, the first layer of the artificial blood vessels comprises endothelium and basement membrane, and the basement membrane comprises pericyte and envelops the endothelium. The endothelium is differentiated from the stem cells expressing surface antigen CD133 or CD34, and the endothelium comprises endothelial cell. The second layer of the artificial blood vessels comprises smooth muscle cell and elastic fiber. The smooth muscle cell is differentiated from the stem cells expressing surface antigen CD133 or CD34. The third layer of the artificial blood vessels comprises, but is not limited to, collagen fiber, connective tissue, small lymphatic vessel, and capillary vessel. The connective tissue comprises fibroblast and macrophage. Therefore, the method in accordance with the present invention enables preparing the artificial vessels with three layers similar to structures, cells and density of human blood vessels comprising endothelium, smooth muscle cell and fibroblast differentiated from the stem cells expressing surface antigen CD133 or CD34.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A method for preparing artificial blood vessels comprising steps of:
preparing a template by 3D printing;
preparing an active polyhedral oligomeric silsesquioxane poly(carbonate-urea) urethane (active POSS-PCU);
preparing stem cells;
mixing the active POSS-PCU with the 1×10⁶ to 2×10⁶ stem cells to form a mixed material containing the stem cells at 4° C.; enveloping the template with the mixed material, and sequentially forming the artificial blood vessels with three layers in a sequential order away from the template; and
processing the artificial blood vessels with three layers by plasma processing, and removing the template to form the artificial blood vessels with an access.

2. The method as claimed in claim 1, wherein the source of the stem cells comprises bone marrow or peripheral blood.

3. The method as claimed in claim 2, wherein the stem cells express surface antigen CD133 or CD34.

4. The method as claimed in claim 1, wherein in the step of mixing the active POSS-PCU with the stem cells, the amount of the stem cells is 1×10⁶.

5. The method as claimed in claim 1, wherein in the step of processing the artificial blood vessels with three layers by plasma processing, conditions of the plasma processing comprise: frequency of DC-discharge plasma 0 Hz, frequency of low/medium-discharge plasma between 10 kilohertz (kHz) and 100 kHz; frequency of radio frequency-discharge plasma 13.56 megahertz (MHz); frequency of microwave-discharge plasma 2.45 gigahertz (GHz); gas flow rate between 0.1 standard liter per minute and 10 standard liters per minute; working power between 1 kilovolt and 40 kilovolts; working power between 1 watt and 180 watts, and reaction time between 5 seconds and 420 seconds.
